(19) United States
(12) Patent Application Publication
(10) Pub. No.: US 2026/0293909 A1
(43) Pub. Date: Oct. 1, 2026

(54) CONTROL OF XYLELLA SP. INFECTION IN PLANTS WITH ACHROMOBACTER VITIS

(71) Applicant: The Regents of the University of California, Oakland, CA (US)

(72) Inventors: Philippe Rolshausen, Riverside, CA (US); M. Caroline Roper, Riverside, CA (US); Elizabeth Deyett, Riverside, CA (US)

(73) Assignee: The Regents of the University of California, Oakland, CA (US)

(21) Appl. No.: 19/490,847

(22) PCT Filed: Jun. 13, 2024
(86) PCT No.: PCT/US2024/033862

§ 371 Date: Dec. 5, 2025

Related U.S. Application Data
(60) Provisional application No. 63/508,402, filed on Jun. 15, 2023.

(51) Int. Cl. A01N 63/20; A01N 25/30; A01P 1/00

(52) U.S. Cl. CPC A01N 63/20; A01N 25/30; A01P 1/00

(57) ABSTRACT

The invention provides compositions comprising Achromobacter vitis and methods of using such compositions for preventing and/or treating infections of a plant with a Xylella sp, e.g., Xylella fastidiosa.

### CROSS-REFERENCE TO RELATED APPLICATIONS

This application is a U.S. National Phase Application of PCT/US2024/033862, International Filing Date Jun. 13, 2024, and which claims priority to U.S. Provisional Application No. 63/508,402, filed Jun. 15, 2023, each of which is herein incorporated by reference in its entirety for all purposes.

### SEQUENCE LISTING

The instant application contains a Sequence Listing which has been submitted electronically in XML format and is hereby incorporated by reference in its entirety. Said XML copy, created on Dec. 5, 2025, is named 081906-1533667-252710US_SL.xml and is 4,423 bytes in size.

### BACKGROUND

Xylella fastidiosa is an aerobic, gram-negative, xylem-limited bacterium of the genus Xylella. The bacterium is a plant pathogen, which has a large host range, including at least 28 families of both monocotyledonous (monocot) and dicotyledonous (dicot) plants.

Plant hosts for X. fastidiosa include, but not limited to grape, oleander, oak, almond, peach, pear, citrus, coffee, maple, apricot, plum, blackberry, mulberry, olive, elm, sycamore, and alfalfa, sweetgum, redbud, hibiscus, catharantus, jacaranda, magnolia, daylily, crape myrtle, dogwood, maidenhair, and Chitalpa tashkentensis plant. Many plant diseases are caused by X. fastidiosa infections, including bacterial leaf scorch, oleander leaf scorch, coffee leaf scorch (CLS), alfalfa dwarf, phony peach disease, and Pierce's disease (PD) of grapes, olive quick decline syndrome (OQDS), and citrus variegated chlorosis (CVC).

X. fastidiosa is exclusively vectored by xylem-feeding insects belonging to the families Cicadellidae and Cercopidae, primarily sharpshooters. The bacterium resides in the xylem. Insects that feed on xylem sap transmit the bacteria from diseased plants to healthy plants and thus are the vector for spread of the disease and bacteria. Plants (e.g., grape plants) shows symptoms of bacterial and infection when the bacteria block the water conducting system and reduce the flow of water to the leaves. The first evidence of PD infection usually is a drying or “scorching” of leaves. About mid-growing season, when foliar scorching begins, some or all of the fruit clusters may wilt and dry up. The bark on affected canes often matures unevenly, leaving islands of mature (brown) bark surrounded by immature (green) bark or the reverse. Environmental stress (e.g., drought) hastens an infected plant's demise.

Current methods of treating or preventing infection by Xylella include control of the insect vectors (such as through pesticide or using physical barriers), destruction of infected plants (such as pruning and freezing), breeding disease-resistant plant varieties, genetically modifying the xylem of the plant to make it uninhabitable for Xylella, using bacteriophages, using broad-spectrum antibiotics, boosting levels of essential plant bacterial micronutrients (such as zinc, iron, copper, and molybdenum that could be toxic to Xylella sp).

### SUMMARY

The disclosure provides a newly identified Acrhomobacter species, Achromobacter vitis, a sample of which is deposited with the American Type Culture Collection (ATCC), 10801 University Blvd., Manassas, VA 20110, United States of America on Mar. 29, 2023 under the conditions of the Budapest Treaty and assigned patent deposit number PTA-127561.

In one aspect, the disclosure provides a method for preventing or treating a plant from Xylella sp. infection comprising inoculating the plant with Achromobacter vitis. In some embodiments, the Xylella sp. is Xylella fastidiosa. In some embodiments, the plant is a monocotyledonous plant. In some embodiments, the plant is a dicotyledonous plant. In some embodiments, the plant is selected from the group consisting of grape, oleander, oak, almond, peach, pear, citrus, coffee, maple, apricot, plum, blackberry, mulberry, olive, elm, sycamore, and alfalfa, sweetgum, redbud, hibiscus, catharantus, jacaranda, magnolia, daylily, crape myrtle, dogwood, maidenhair, and Chitalpa tashkentensis plant. In certain embodiments, the plant is a grape plant, an oleander plant, a coffee plant, an alfalfa plant, a peach plant, an olive plant, or a citrus plant. In particular embodiments, the plant is a grape plant.

In some embodiments, the Achromobacter vitis can be formulated with at least one carrier when inoculating into a plant. In some embodiments, the carrier is selected from the group consisting of a surface-active agent (surfactant), an inert material, a preservative, a humectant, a feeding stimulant, an attractant, an encapsulating agent, a binder, an emulsifier, a dye, a UV protective, a buffer, a flow agent, a liposome, a dispersion medium, a micronutrient donor, and the like.

In some instances, the carrier is a surface-active agent. As disclosed herein, the surface-active agent can be an anionic agent, a cationic agent, or a non-ionic agent. In some embodiments, the anionic surface-active agent is selected from the group consisting of a carboxylate of a metal; carboxylate of a long chain fatty acid; an N-acylsarcosinate; mono or di-esters of phosphoric acid with fatty alcohol ethoxylates or salts of such esters; sodium dodecyl sulfate; sodium octadecyl sulfate; sodium cetyl sulfate; ethoxylated fatty alcohol sulfates; ethoxylated alkylphenol sulfates; lignin sulfonates; petroleum sulfonates; alkyl-benzene sulfonates, butyl-naphthalene sulfonate; salts of sulfonated naphthalene-formaldehyde condensates; salts of sulfonated phenol-formaldehyde condensates; the amide sulfonates, and the dialkyl sulfosuccinates. In some embodiments, the cationic surface-active agent is selected from the group consisting of an aliphatic monoamine, an aliphatic diamine, an aliphatic polyamine; oxygen-containing amine; and an amide-linked amine prepared by the condensation of a carboxylic acid with a di- or polyamine; or a quaternary ammonium salt. In some embodiments, the non-ionic surface-active agent is selected from the group consisting of condensation products of fatty acid esters, fatty alcohols, fatty acid amides, fatty-alkyl- or alkenyl-substituted phenols with ethylene oxide, fatty esters of polyhydric alcohol ethers, condensation products of such esters with ethylene oxide, block copolymers of ethylene oxide and propylene oxide, acetylenic glycols, and ethoxylated acetylenic glycols.

In other instances, the carrier is an inert material. In some embodiments, the inert material is selected from the group consisting of inorganic minerals, phyllosilicates, carbonates, sulfates, phosphates, botanical materials, peanut hulls, rice hulls, and walnut shells.

In some embodiments, inoculating a plant comprises introducing the Achromobacter vitis into the plant, e.g., introducing the Achromobacter vitis into a xylem of the plant. In some embodiments, inoculating comprises introducing the Achromobacter vitis into the plant by injection, spraying, misting, dusting, or topical administration to the plant. In some instances, the Achromobacter vitis is injected into the plant. In other instances, the Achromobacter vitis is sprayed into the plant. In some embodiments, the plant is inoculated with Achromobacter vitis before the onset of at least one symptom of Xylella infection. In other instances, a plant is treated with Achromobacter vitis after at least one symptom indicative of Achromobacter vitis infection appears in the plant. In some embodiments, the symptom is leaf scorch, leaf chlorosis, gum-like substance on leave, withering and desiccation of branches, dwarfing or lack of growth of the plant, drooping appearance and shorter internodes, shriveled fruits on infected plants, premature fruit abscission, hardening and size reduction of fruits, wilting of foliage, or browning to dieback.

In particular embodiments, the plant is a grape plant and is treated with Achromobacter vitis, e.g., by spraying, at the onset of one or more symptoms of Pierce's disease. In other instances, the grape plant may be treated before visible symptoms appear. Pierce's disease symptoms can include, but are not limited to, leaf scorch, yellowing around the leaf margins or between the veins, leaves that are smaller and less vigorous, and patches of brown and green tissue (green islands) on stems.

In some embodiments, the method further comprising inoculating the plant with a biological or chemical agent that inhibits Xf infection. In some embodiments, the agent comprises a microorganism, an extract of a microorganism, a pesticide, a bacteriophage, an antibiotic drug, zinc, iron, copper, molybdenum, a derivative thereof, or a combination thereof.

The present disclosure also provides a method of preparing an environment for growth of a plant susceptible to Xylella sp. infection comprising inoculating the plant with the bacterium Achromobacter vitis. In some embodiments, the Achromobacter vitis is a strain deposited under ATCC Accession No. PTA-127561. In some embodiments, the Xylella sp. is Xylella fastidiosa. In some embodiments, the plant is a monocotyledonous plant. In some embodiments, the plant is a dicotyledonous plant. In some embodiments, the plant is a grape, oleander, oak, almond, peach, pear, citrus, coffee, maple, apricot, plum, blackberry, mulberry, olive, elm, sycamore, alfalfa, sweetgum, redbud, hibiscus, catharantus, jacaranda, magnolia, daylily, crape myrtle, dogwood, maidenhair, or Chitalpa tashkentensis plant. In certain embodiments, the plant is a grape plant, an oleander plant, a coffee plant, an alfalfa plant, a peach plant, an olive plant, or a citrus plant. In particular embodiments, the plant is a grape plant.

In some embodiments, the plant is inoculated with Achromobacter vitis by spraying, misting, or dusting the Achromobacter vitis into or on soil in which the plant is growing or into or on soil prior to planting the plant in the soil. In some embodiments, the plant is inoculated with Achromobacter vitis by introducing the Achromobacter vitis into irrigation water that is provided to the plant.

The present disclosure further provides a composition for treating or preventing a Xylella sp. infection in a plant, the composition comprising Achromobacter vitis and at least one carrier formulated for delivery to the plant. In some embodiments, the Achromobacter vitis is the strain deposited under ATCC Accession No. PTA-127561. In some embodiments, the carrier is selected from the group consisting of a surface-active agent (surfactant), an inert material, a preservative, a humectant, a feeding stimulant, an attractant, an encapsulating agent, a binder, an emulsifier, a dye, a UV protective, a buffer, a flow agent, a liposome, a dispersion medium, a micronutrient donor, and the like.

In some embodiments, the surface-active agent is an anionic agent, a cationic agent, or a non-ionic agent. In some embodiments, the anionic surface-active agent is selected from the group consisting of a carboxylate of a metal; carboxylate of a long chain fatty acid; an N-acylsarcosinate; mono or di-esters of phosphoric acid with fatty alcohol ethoxylates or salts of such esters; sodium dodecyl sulfate; sodium octadecyl sulfate; sodium cetyl sulfate; ethoxylated fatty alcohol sulfates; ethoxylated alkylphenol sulfates; lignin sulfonates; petroleum sulfonates; alkyl-benzene sulfonates, butyl-naphthalene sulfonate; salts of sulfonated naphthalene-formaldehyde condensates; salts of sulfonated phenol-formaldehyde condensates; the amide sulfonates, and the dialkyl sulfosuccinates. In some embodiments, the cationic surface-active agent is selected from the group consisting of an aliphatic monoamine, an aliphatic diamine, an aliphatic polyamine; oxygen-containing amine; and an amide-linked amine prepared by the condensation of a carboxylic acid with a di- or polyamine; or a quaternary ammonium salt. In some embodiments, the non-ionic surface-active agent is selected from the group consisting of condensation products of fatty acid esters, fatty alcohols, fatty acid amides, fatty-alkyl- or alkenyl-substituted phenols with ethylene oxide, fatty esters of polyhydric alcohol ethers, condensation products of such esters with ethylene oxide, block copolymers of ethylene oxide and propylene oxide, acetylenic glycols, and ethoxylated acetylenic glycols.

In some embodiments, the inert material is inorganic minerals, phyllosilicates, carbonates, sulfates, phosphates, botanical materials, peanut hulls, rice hulls, and walnut shells.

In some embodiments, the composition further comprises a biological or chemical agent which inhibits Xf infection. In some embodiments, the agent comprises a microorganism, an extract of a microorganism, a pesticide, a bacteriophage, an antibiotic drug, zinc, iron, copper, molybdenum, or a derivative thereof or combination thereof.

The disclosure also provides an in vitro culture of isolated Achromobacter vitis, a representative sample of which is deposited under ATCC Accession No. PTA-127561.

### BRIEF DESCRIPTION OF THE DRAWINGS

FIG. 1 shows a maximum likelihood phylogeny tree of 14 Achromobacter taxa and closely related groups. The branch support represents how often the branch occurred out of 1000 resampling events. Achromobacter vitis UCR-ACH1 is the sole member of a unique branch within the Achromobacter genomes analyzed representing a novel species within this genus.

FIG. 2A-C shows that PD symptom severity is reduced in vines pretreated with endophytes compared to vines pretreated with 1×PBS. A. Severity of PD symptoms on Cabernet Sauvignon grapevines pretreated with Pa. phytofirmans (PsJN), Achromobacter vitis (ACH), or Pseudomonas viridiflava (PV) or 1×PBS and then challenged with X. fastidiosa either 1 or 7 days later. Boxplots represent the median and variation of the disease rating scores (two biological replicates of approximately 8-10 technical replicate plants) at 12 weeks post inoculation for 1 day vines and 15 weeks post-inoculation for 7 day vines. Gray bars reflect the disease rating at which vines will no longer survive the season (Disease rating of 2 or more). B. The percentage of survivability of vines over time, where vines scoring a disease rating of 2 or higher will not survive the season. Solid lines represent vines challenged with X. fastidiosa 1 day after biocontrol inoculation, where data were collected over the course of 6 weeks. Dotted lines represent vines challenged with X. fastidiosa 7 days after biocontrol inoculation, where data were collected over the course of 10 weeks. C. Cox proportional hazard analysis, a survival analysis regression model, showed each endophyte confer a significant reduction in risk of death when inoculated with X. fastidiosa regardless of the timing X. fastidiosa challenge (Cox Proportional hazards; p<0.001).

FIG. 3 shows that X. fastidiosa DNA concentration measured from the petioles of Cabernet Sauvignon grapevines pretreated with endophytes is reduced compared to vines pretreated with 1×PBS. Boxplots represent the median and variation of X. fastidiosa titer (fg X. fastidiosa DNA per ng of plant DNA extracted) from each of 18-20 replicate plants (two biological replicates of 8-10 technical replicate plants). X. fastidiosa titer was significantly reduced in vines pretreated with Pa. phytofirmans (PsJN), Achromobacter vitis (ACH), and Pseudomonas viridiflava (PV) compared to pretreatment with 1×PBS at the day 1 challenge. X. fastidiosa titer was significantly reduced in vines pretreated with Pa. phytofirmans and Achromobacter vitis compared to pretreatment with 1×PBS at the day 7 challenge, but not Pseudomonas viridiflava.

FIG. 4 shows that viable populations of endophytes persist in Cabernet Sauvignon grape stems over time. Viable population sizes of Pa. phytofirmans (PsJN), Achromobacter vitis (ACH), and Pseudomonas viridiflava (PV) in Cabernet Sauvignon grape stems at the point of inoculation (POI), 3 internodes above the POI, and 7 nodes above the POI. Horizontal rows represent the internode sampled and vertical columns represent the endophyte. Circles represent bacterial population sizes and lines represent the regression line from three plants measured at 5 times points.

FIG. 5a-b shows inhibition of growth of X. fastidiosa in the presence of endophytes. FIG. 5a. shows 1×PBS (section A), Pa. phytofirmans PsJN (section B), Achromobacter vitis (section C), and Pseudomonas viridiflava (section D) were plated against 5 μL droplets of X. fastidiosa. FIG. 5b. shows bar plots represent average distance from Pa. phytofirmans (PsJN), Achromobacter vitis (ACH), and Pseudomonas viridiflava (PV) to the first sign of X. fastidiosa growth on solid PD3 plates. Vertical bars represent the standard error of the average distance of inhibition from 9 replicate plates.

### DETAILED DESCRIPTION

### I. Introduction

Xylella fastidiosa is a re-emerging plant pathogen that causes disease of several vine and tree crops. However, there are no treatments targeting the pathogen itself.

Achromobacter has been identified as a member of the grapevine microbiome and is a known biocontrol agent in several pathosystems (Deyett et al., 2017; Moretti et al., 2008; Niem et al., 2020, US20200000099 A1). The present disclosure provides a new species of Achromobacter, Achromobacter vitis UCR-ACH1, which was originally isolated from grapevine grown in Temecula, California. This new species can be used as a biological control agent to prevent or treat infection of a plant with a Xylella sp., e.g., Xylella fastidiosa. Accordingly, the present disclosure further provides methods of preventing and/or treating plants with Achromobacter vitis, or of preparing an environment for growth of a plant susceptible to Xylella sp. infection by using Achromobacter vitis.

### II. Terminology

As used herein, the singular forms “a,” “and” and “the” include plural referents unless the context clearly dictates otherwise. Thus, for example, reference to “a plant” includes a plurality of such plants and reference to “a pathogen” includes reference to one or more pathogens.

The use of “or” means “and/or” unless stated otherwise. Similarly, “comprise,” “comprises,” “comprising” “include,” “includes,” and “including” are interchangeable and not intended to be limiting.

The terms “about” and “approximately” generally mean an acceptable degree of error for the quantity measured given the nature or precision of the measurements. Typical, exemplary degrees of error are within 20 percent (%), preferably within 10%, or within 5% of a given value or range of values. Numerical quantities given herein are approximate unless stated otherwise.

It is to be further understood that where descriptions of various embodiments use the term “comprising,” those skilled in the art would understand that in some specific instances, an embodiment can be alternatively described using language “consisting essentially of” or “consisting of.”

As used herein, the term “prevent” or “preventing” a Xylella sp. infection (e.g., an Xylella fastidiosa infection) in a plant refers to protecting a plant from infection with a Xylella sp., or decreasing the risk that a plant can be infected by the Xylella sp.

As used herein, the term “treat” or “treating” a Xylella sp. infection, e.g., an Xylella fastidiosa infection, in a plant refers to the reduction in, eradication of, or delay of onset or progression of one or more symptoms caused by the Xylella sp. infection by methods described herein. General symptoms exhibited by a plant infected with a Xylella sp. include, but are not limited to, leaf scorch, leaf chlorosis, gum-like substance on leave, withering and desiccation of branches, dwarfing or lack of growth of the plant, drooping appearance and shorter internodes, shriveled fruits on infected plants, premature fruit abscission, hardening and size reduction of fruits, wilting of foliage, browning to dieback and death. Symptoms of grapevine infection is Xylella sp. include, but are not limited to, leaf scorch, yellowing around the leaf margins or between the veins, less vigorous and smaller leaf, and patches of brown and green tissue (green islands) on stems. In some embodiments, the disclosed methods may not necessarily result in eradication or cure of the Xylella sp. infection (e.g., an Xylella fastidiosa infection) in a plant, but can significantly reduce the symptoms caused by the infection.

The term “effective amount” or “sufficient amount” refers to the amount of the composition that is sufficient to effect beneficial or desired results, e.g., alleviating one or more symptoms of infection, reducing the rate of progression of the infection and the like. The effective amount may vary depending upon one or more of: the plant and pathogen condition being treated, the severity of the infection, the manner of administration and the like, which can readily be determined by one of ordinary skill in the art. The specific amount may vary depending on one or more of: the particular formulation agent chosen, the plant species, the location of the Xf infection in the plant, whether it is administered in combination with other compounds, and timing of administration.

The term “carrier” refers to a substance that aids the administration of an active agent (such as Achromobacter vitis) to a subject (such as a plant). A carrier can be included in the compositions of the disclosure that causes no significant adverse toxicological effect to the plant. Non-limiting examples of carriers include a surface-active agent, an inert material, a preservative, a humectant, a feeding stimulant, an attractant, an encapsulating agent, a binder, an emulsifier, a dye, a UV protective, a buffer, a flow agent, a liposome, a dispersion medium, a micronutrient donor, and the like. For examples, a carrier may be substances for providing the formulation with stability and isotonicity. In some instances, the carrier is an agent that facilitates the delivery of Achromobacter vitis to a plant.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### III. Detailed Description of the Embodiments

Xylella fastidiosa (Xf) is an aerobic, gram-negative, xylem-limited bacterium that dwells in a wide array of plant species. Both monocotyledonous and dicotyledonous plants can be infected by X. fastidiosa. Plant susceptible to Xf infection, include, but not limited to grape, citrus, oleander, oak, almond, peach, pear, coffee, maple, apricot, plum, blackberry, mulberry, olive, elm, sycamore, alfalfa, sweetgum, redbud, hibiscus, catharantus, jacaranda, magnolia, daylily, crape myrtle, dogwood, maidenhair, and Chitalpa tashkentensis plant.

The present disclosure provides a newly identified anti-Xf bacterial species, Achromobacter vitis, which was originally isolated from grapevine wood tissue. Achromobacter vitis can inhibit Xylella sp. growth in vivo and mitigate disease symptom appearance. The present disclosure thus also provides methods and compositions using Achromobacter vitis for preventing or treating Xylella sp., e.g., Xylella fastidiosa, infection in plants.

A. Characterization of Achromobacter vitis

As disclosed herein, Achromobacter vitis comprises a genome sequence that has at least 95% or 96% nucleotide sequence identity; or at 97%, 98%, or 99%, nucleotide sequence identity, to the genomic sequence deposited under GenBank Accession No. SAMN26814322, or to the genomic sequence of the bacteria deposited with ATCC under Patent Deposit No. PTA-127561.

In some embodiments, the genome of such an Achromobacter vitis species is about 6.4-6.5 million bp of nucleic acids, or about 6.5 million bp in length. In particular embodiments, the Achromobacter vitis genome comprises about 6,489,685 bp of nucleic acids in length. The Achromobacter vitis genome typically comprises about 66% GC content and contains about 5,877 coding regions.

In one aspect, the present disclosure provides an in vitro culture of Achromobacter vitis. The Achromobacter vitis can grow in vitro in any suitable medium, including liquid medium (such as a broth medium), semi-solid medium or a solid medium (such as an agar medium).

Achromobacter vitis can be cultured using methods suitable for the culture of other Achromobacter species. For example, medium for culturing Achromobacter vitis include, but are not limited to, Tryptic Soy Agar (TSA), Potato Dextrose Agar (PDA), Luria-Bertani (LB), Terrific broth (TB), M9 minimal medium (M9), S. O. C medium, or 2-YT. In particular embodiments, the Achromobacter vitis grows on Tryptic Soy Agar (TSA). Cultures can be grown, for example, between about 20-35° C., typically, from about 25° C. to 30° C.

B. Preventing or Treating Xylella sp. Infection Using Achromobacter vitis

The present disclosure provides methods and compositions using Achromobacter vitis for preventing or treating Xylella sp. infection in plants. In some embodiments, Achromobacter vitis can be used to prevent infection or to protect a plant from a Xylella sp. infection. In some embodiments, Achromobacter vitis can be used to prepare an environment for growth of a plant susceptible to a Xylella sp. infection. In some embodiments, Achromobacter vitis can be used to treat a Xylella sp. infection in a plant. In preferred embodiments, the method and compositions are used to treat or prevent Xylella fastidiosa infection.

The methods disclosed herein for preventing or treating Xylella sp. infection in a plant comprise applying an effective amount of a composition comprising the Achromobacter vitis. As disclosed herein, “effective amount” is intended to mean an amount sufficient to control Xylella sp. infection in a plant. The effective amount may vary depending upon one or more of: the plant and pathogen condition being treated, the severity of the disease condition, the manner of administration and the like, which can readily be determined by one of ordinary skill in the art. The specific amount may vary depending on one or more of: the particular formulation agent chosen, the plant species, the location of the Xf infection in the plant, whether it is administered in combination with other compounds, and timing of administration. In some embodiments, the composition comprises at least about 10⁴ Colony Forming Unit (CFU)/milliliter (CFU/ML) of Achromobacter vitis in one inoculation for preventing or treating Xylella sp infection in a plant. In some embodiments, the composition comprises no more than about 10⁸ CFU/ml of Achromobacter vitis in one inoculation. In particular embodiments, the composition comprises about 10⁴-10⁸ CFU/ml of Achromobacter vitis each inoculation.

Achromobacter vitis is typically administered in an aqueous solution, e.g., as a liquid injected into or sprayed onto a plant. Composition for applying Achromobacter vitis to a plant may further comprise additional agent(s) to facilitate delivery or treatment of the plant. In some embodiments, a composition may comprise a carrier formulated for delivery Achromobacter vitis to a plant. In some embodiments, the carrier is selected from the group consisting of a surface-active agent (surfactant), an inert material, a preservative, a humectant, a feeding stimulant, an attractant, an encapsulating agent, a binder, an emulsifier, a dye, a UV protective, a buffer, a flow agent, a liposome, a dispersion medium, a micronutrient donor, and the like.

In some instances, the carrier is a surface-active agent. Suitable surface-active agents include an anionic agent, a cationic agent, or a non-ionic agent. Anionic surface-active agents include, but are not limited to, a carboxylate of, for example, a metal; carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

In some embodiments, treatment with Achromobacter vitis may comprise treating the plant with an additional agent to facilitate product handling and application to a plant or help plant growth and harvest. In some embodiments, agent is a plant growth regulator or fertilizer. In some embodiments, the compositions may comprise an agrochemical, such as a herbicide, insecticide, nematicide, molluscicide, or acaracide.

In some embodiments, Achromobacter vitis is mixed with another agent, e.g., chemical or biological agent(s), in the same formulation and delivered by the same carrier. In other embodiments, the Achromobacter vitis and the chemical or biological agent(s) are in the different formulations and delivered by different carriers. The Achromobacter vitis of the disclosure may be applied simultaneously or in succession with other compounds.

Compositions comprising Achromobacter vitis, can be applied directly to the plant, or part of a plant to be treated. In other embodiments, compositions are applied to the environment, e.g., to the soil. Application can be performed during growth, seeding or storage.

In some embodiments, the Achromobacter vitis is inoculated into the plant together with at least one additional biological or chemical agent. In some embodiments, the biological or chemical agent is an anti-Xf agent which inhibits Xf infection in a plant. In some instances, the anti-Xf agent is a biological agent, including but not limited to, a microorganism such as a bacterial or fungal species, or chemical agent or extract prepared from the bacterial or fungal species, as disclosed in US20200000099; or a bacteriophage. In other instances, the anti-Xf agent is a chemical agent, including but not limited to, a pesticide, an antibiotic drug, zinc, iron, copper, molybdenum, a derivative thereof, or a combination thereof.

Compositions comprising Achromobcter vitis can be in a suitable form for direct application or initially formulated as a concentrate that requires dilution with a suitable quantity of water or other diluent before application. The compositions of the disclosure find use in protecting plants, seeds, and plant products in a variety of ways. For example, the compositions can be used in a method that involves placing an effective amount of the composition in the environment of the pathogen by procedures including injection, spraying dusting, coating, and the like.

C. Inoculation of Achromobacter vitis

Compositions comprising Achromobacter vitis can be applied to the environment of a plant Xylella pathogen by, for example, injecting, spraying, atomizing, misting, dusting, scattering, coating, pouring, or other topical administration to a plant. The compositions of the disclosure can be introduced into or on the soil, into irrigation water, by seed treatment or general application. The compositions of the disclosure can be introduced at the time when the pathogen has begun to appear or before the appearance of pathogens as a protective measure. It is generally important to obtain good control of pathogens in the early stages of plant growth, as this is the time when the plant can be most severely damaged. The compositions of the disclosure can conveniently contain an insecticide if this is thought necessary.

The methods disclosed herein include an inoculation of the Achromobacter vitis to the plant. The Achromobacter vitis can be delivered or introduced to the plant directly or indirectly. As used herein, “inoculation” refers to any method of delivering Achromobacter vitis to the plant. In some instances, the plant is inoculated with Achromobacter vitis by direct introduction into the plant e.g., by injection into xylem of the plant. For example, a syringe needle can be attached to a syringe barrel and used to pierce the base of the vine with the needle. The injection apparatus can be secured to the vine and provide an instant delivery of the formulation or left in place to provide a continuous drip of the formulation into the xylem stream of the plant. In other instances, Achromobacter vitis is introduced to the plant through soil or water in which the plant grows. In some embodiments, the inoculation comprises introducing or delivering the Achromobacter vitis into the plant by injection, spraying, misting, dusting, or topical administration to the plant. In a particular embodiment, Achromobacter vitis is sprayed onto the plant.

The present disclosure also provides a method of preparing an environment for growth of a plant susceptible to Xylella sp. infection comprising inoculating the plant with Achromobacter vitis. In some embodiments, the Achromobacter vitis is introduced to the plant through soil or water in which the plant grows. In some instances, the inoculating comprises spraying, misting, or dusting the Achromobacter vitis into or on soil that the plant grows. In other instances, the inoculating comprises introducing the Achromobacter vitis into irrigation water that apply to the plant.

### D. Plants

The anti-Xf endophytic Achromobacter vitis of the disclosure can be used for the prevention or treatment of a Xylella sp. infection of any plant vulnerable to such an infection. In preferred embodiments, Achromobacter vitis is used to treat or prevent Xylella fastidiosa infection. In some embodiments, the plant is a monocotyledonous plant. In other embodiments, the plant is a dicotyledonous plant. In some embodiments, a plant that is treated with Achromobacter vitis is a grape, oleander, oak, almond, peach, pear, citrus, coffee, maple, apricot, plum, blackberry, mulberry, olive, elm, sycamore, alfalfa, sweetgum, redbud, and hibiscus, catharantus, jacaranda, magnolia, daylily, crape myrtle, dogwood, maidenhair, Chitalpa tashkentensis, or a forest tree species plant susceptible to Xylella sp. infection. In certain embodiments, the plant is a grape, citrus, oleander, coffee, alfalfa, peach, or olive plant, or an ornamental plant. In particular embodiments, the plant is grape.

The plant may be at any stage of growth, including seeds, seedlings, or full plants. In addition, as discussed herein, any part of the plant or the soil or water may be inoculated by Achromobacter vitis or a composition comprising Achromobacter vitis. Suitable plant parts include, but not limited to, seeds, roots, leaves, flowers, stems, trunks, etc. In certain embodiments, the delivery of Achromobacter vitis or a composition comprising Achromobacter vitis is to the endosphere or trunk of the plant. In a particular embodiment, the delivery is to a xylem of the plant.

### IV. Examples

The present disclosure will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1: Genome Sequence Data of Achromobacter vitis

Achromobacter vitis UCR-ACH1 was isolated from grapevine stem. Grapevine cultivar ‘Syrah’ on 1103P rootstock was located in a commercial vineyard planted in 2010 in Temecula, California. This vineyard expressed a range of Pierce's disease (PD) symptoms from asymptomatic to very symptomatic. Lignified stems from a PD-asymptomatic grapevine were harvested and brought back to the laboratory at UC Riverside. Stem tissues were surface sterilized and cultured on Potato Dextrose Agar (PDA). Pure isolate of Achromobacter vitis UCR-ACH1 was cultured on Tryptic Soy Agar (TSA) at 28° C. for 2 days and stored on Tryptic Soy Broth (TSB) with 15% glycerol at −80° C.

A single colony of the isolated Achromobacter vitis UCR-ACH1 was inoculated in TSB overnight at 28° C. with constant shaking. Genomic DNA was purified using the Wizard Genomic DNA Purification Kit (Promega Corp., Madison, WI, USA) according to manufacturer's instructions. DNA was submitted for long read (Nanopore) and short read (Illumina) sequencing to the Microbial Genome Sequencing Center (MIGS, Pittsburgh, PA). A total of 5.3 million reads for the Achromobacter vitis UCR-ACH1 was generated (Table 1).

TABLE 1
Genome features of the endophytic Achromobacter vitis UCR-
ACH1 isolated from grapevine wood tissue.

| Features | Achromobacter vitis UCR-ACH1 |
|---|---|
| BioSample Accession | SAMN26814322 |
| Source and Location | Grapevine, Temecula, California |
| No. of Reads | 4214781 |
| % GC | 66 |
| Assembly Size (bp) | 6489685 |
| N50 | 6489685 |
| No. of Contigs | 1 |
| No. of Coding Genes | 5877 |
| GTDB Assign Taxon | Achromobacter deleyi GCF_902859705.1 |
| GTDB FastANI Score | 94.33 |

Porechop v.0.2.4 (Wick et al., 2017), Filtlong v.0.2.1 (https site github.com/rrwickFiltlong), and Trimmomatic v.0.39 (Bolger et al., 2014) were used to perform quality control and adapter trimming on ONT and Illumina sequences, respectively. A hybrid assembly of short reads and long reads was built using Unicycler v.0.4.4 (Wick et al., 2017). Assemblies were assessed with QUAST v.5.0.2 (Gurevich et al., 2013) and annotated using Prokka v.1.14.6 (Seemann, 2014). Taxonomy of genomes was determined through the GTDB-Tk database (Chaumeil et al., 2019) and a maximum likelihood tree via the KBase platform (Arkin et al., 2018). To further profile the Achromobacter vitis strain, a maximum likelihood tree was constructed using KBase with a subset of 49 orthologous gene domain clusters. The resulting tree was visualized in Interactive Tree of Life (Letunic and Bork, 2019).

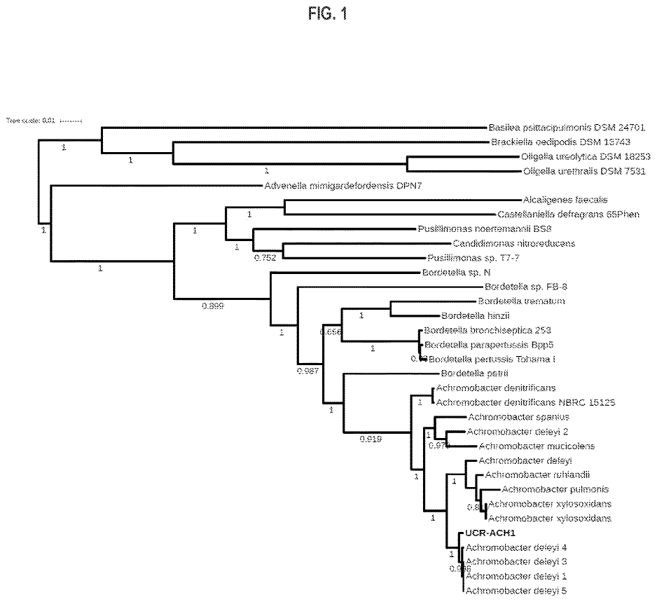

The Achromobacter vitis UCR-ACH1 assembly consisting of 1 contig of 6,489,685 bp. The closest species to this assembled genome was found to be Achromobacter deleyi via GTDB-tk, with a FastANI score of 94.33%, which is lower than the recommended species cut-off of 95%. Thus, to further assign taxonomic identity, a maximum likelihood tree was constructed using KBase with a subset of 49 orthologous gene domain clusters (FIG. 1). The tree supports the identification given by the GTDB-Tk database. Achromobacter vitis UCR-ACH1 falls between Achromobacter spanius and Achromobacter deleyi but remains on its own branch representing a novel Achromobacter species.

The metabolic profiling through DRAM v.1.2.4 (Shaffer et al., 2020) and antiSMASH 6.0 (Blin et al., 2021) revealed the genome contains genes for siderophore, nitrogen fixation, 1-aminocyclopropane-1-carboxylate, and 1-aminocyclopropane-1-carboxylate deaminase, which have all been shown to contribute to microbe-microbe antagonism or to benefit the host through growth promotion or induced systemic resistance (Ali et al., 2021). The Achromobacter vitis UCR-ACH1 assembly contained beta lactone (antagonism), ectoine (osmotic stress supporter), and anthranilate (biofilm disruptor). Thus, the genome contains genes related to mechanisms of biocontrol in other bacterial systems. This genome could contribute to a broader molecular understanding of biocontrol agents in agriculture.

### Data Availability

The complete genome sequence and associated data for Achromobacter vitis UCR-ACH1 are deposited under GenBank biosample SAMN26814322. Short Read Archive accession numbers are SRR18390886, SRR18390887. A representative sample of Achromobacter vitis is deposited under ATCC Accession No. PTA-127561.

### Example 2: Antibiosis Activity of Achromobacter vitis

### Materials and Methods

Identification of Biocontrol Agents. Two bacterial strains were assessed as biocontrol agents in this study: a Pseudomonas viridiflava UCR-PV1 and an Achromobacter vitis UCR-ACH1. These strains were isolated from grapevine hosts in California and were chosen based on negative correlation to disease symptom severity and pathogen titer (Deyett et al., 2017). Pa. phytofirmans strain PsJN is a biocontrol agent for PD of grapevine that was originally isolated from onion and was used as a positive control for biocontrol efficacy in our bioassays (Baccari et al. 2019). The Pa. phytofirmans, Pseudomonas viridiflava, and Achromobacter vitis were grown on Tryptic Soy Agar (TSA) for 24 h at 28° C. X. fastidiosa subsp. fastidiosa strain Temeculal was grown on PD3 medium for 5 days at 28° C. according to standard methods (Roper et al. 2007).

PD bioassays. For in planta assays, Vitis vinfera cv. Cabernet Sauvignon one-bud cuttings were rooted (generously provided by Foundation Plant Services, University of California, Davis) in vermiculite. Rooted cuttings were potted in UCR soil mix and amended with 5 g of controlled release fertilizer (Scotts Osmocote Classic, N-P-K: 14-14-14). Vines were propagated in the greenhouse and trained vertically as a single shoot onto a stake.

Inoculations were prepared as follows: cells were harvested from 5-day old plates and suspended in sterile 1×PBS and adjusted to an OD₆₀₀=0.25. For each trial, 10 plants were inoculated with one of 4 treatments: Pa. phytofirmans, Pseudomonas viridiflava, Achromobacter vitis, or 1×PBS. Plants were inoculated with 10 μL of biocontrol agent or 1×PBS solution between the first and second node of the plant on both sides by mechanical needle inoculation as previously described (Hill et al., 1995). Ten μL of X. fastidiosa suspension was needle-inoculated into the plant between the second and third node (above the biocontrol inoculant) on one side of the stem, at 1 day or 7 days post biocontrol agent inoculation. A randomized block design was utilized. Visual PD severity symptom ratings (0=healthy vine, 1=one or two leaves with scorching at the margins, 2=two or three leaves with more developed scorching, 3=all the leaves have some scorching with a few matchstick petioles, 4=all leaves have heavy scorching and many matchstick petioles, and 5=dead vine) (Deyett et al., 2019) were taken through sample collection occurring when 1×PBS-inoculated control vines reached an average disease rating of 3. The experiment was repeated on a different set of plants.

Detection and quantification of X. fastidiosa in planta. Detection and quantification of X. fastidiosa was determined using the method described by Deyett et al. (2019). Briefly, three petioles were harvested from the point of inoculation (POI) and pooled. Petioles were frozen, lyophilized for 36 h with a FreeZone 2.5 L benchtop freeze dry system (Labconco, Kansas City, MO) and ground to a powder at room temperature using a MM300 grinder (Retsch, Bucks County, PA; 45 s 25 oscillations per second) in a 35 mL stainless-steel grinding jar (Retsch, Bucks County, PA) with 20 mm stainless steel balls. DNA was extracted from 15 mg of ground petiole tissue using the ZymoBIOMICS DNA miniprep kit per manufacturer's protocol (Zymo Research, Irvine, USA). DNA was quantified using a Qubit 2.0 fluorometer (Invitrogen, Waltham, MA) according to the manufacturer's protocol.

X. fastidiosa ITS primers: XfITSF6: 5′-GAGTATGGTGAATATAATTGTC-3′ (SEQ ID NO: 1) and XfITSR6: 5′-CAACATAAACCCAAACCTAT-3′ (SEQ ID NO: 2) were utilized to quantify pathogen abundance in each sample using quantitative PCR (qPCR). qPCR was performed in a CFX96 Real-Time PCR cycler using CFX Maestro V. 1.1 software (Bio-Rad) using default settings for amplification curve analysis. Reactions occurred at a final volume of 25 μl of 12.5 μL of Quantitect SYBR Green Master Mix reagent (Qiagen, Germantown, MD), 0.4 μM of each primer, and 2 μL of DNA template. The cycling program consisted of 95° C. for 15 min: 40 cycles of 55° C. for 15 s, 72° C. for 45 s, 95° C. for 15 s; and a melt curve analysis of 65-95° C. (0.5° C. increments every 5 s). Each qPCR plate was accompanied with both sterile water as a negative control and a standard curve consisting of four tenfold dilutions of 6 ng of X. fastidiosa DNA. Each sample was tested in triplicate.

Quantitative assessment of temporal and spatial distribution of biocontrol strains in planta. For in planta assessment of biocontrol presence and movement, vines and bacterial cultures were prepared as described for the PD bioassays. Plants were inoculated with 10 μL of an individual biocontrol agent suspension or 1×PBS solution between the first and second node of the plant on both sides by mechanical needle inoculation (Hill et al., 1995). Three plants were used for each treatment at each time point of 2, 4, 8, 12, 16 weeks. Thus for 4 treatments at five different time points 75 plants were utilized. Plants were grown under greenhouse conditions for 16 weeks. The experiment was repeated on a different set of plants.

At each time point (2, 4, 8, 12, 16 weeks), stem samples were destructively sampled at the point of inoculation (POI), 3 nodes above the POI, and 7 nodes above the POI. Each sample was surface sterilized in 70% ethanol for five minutes and rinsed three times in sterile distilled water. The samples were placed in individual mesh lined, sample bags (Agdia, Inc., Elkhart, IN) and homogenized with a hammer in 2 mL 1×PBS. 10 μL from each resulting slurry solution was plated on Tryptic Soy Agar and allowed to grow at 28° C. for 48 hours. Colony counts were recorded and normalized to colony forming units per gram of tissue (cfu/g), and biocontrol identity was confirmed via Sanger sequencing with 16S universal primers: 16S U1: 5′-CCAGCAGCCGCGGTAATACG-3′ (SEQ ID NO: 3) and 16S U2: 5′-ATCGGCTACCTTGTTACGACTTC-3′ (SEQ ID NO: 4).

In vitro inhibition assay. To determine if the Pa. phytofirmans, Pseudomonas viridiflava, and Achromobacter vitis inhibited X. fastidiosa through an antibiosis mechanism, the inventors utilized a dot inhibition assay. Briefly, inocula of the individual biocontrol agents, the Pa. phytofirmans, Pseudomonas viridiflava, and Achromobacter vitis, of OD₆₀₀=0.25 were prepared in sterile 1×PBS buffer. Inoculum of X. fastidiosa of OD₆₀₀=0.25 was also prepared in sterile 1×PBS buffer. A 10 μL drop of each inocula was plated on PD3 agar plates and spread across a length of 5 cm by gently tilting the plates. Single drops of 5 μL of X. fastidiosa inoculum were plated 1, 2, 3, 4, and 5 cm away from the endophyte drip line. The inocula were allowed to air dry in a biosafety cabinet and incubated at 28° C. for 7 days and observed for growth. The distance to the first sign of growth was recorded.

Statistical Analyses. All statistical analyses and graph visualizations were done using R v. 4.6.1 (http://www.R-project.org/). Differences in PD visual symptoms and pathogen quantities were determined through Kruskal-Wallis rank sum statistical rating and pairwise Wilcoxon test was also used to calculate the differences between both biocontrol agents and time periods. P-values were adjusted with a false discovery rate. Parametric survival model using log normal distributions with post hoc Tukey HSD test was conducted to determine the differences in survival between biocontrol agents and analysis was adapted from Schandry (2017).

### Results

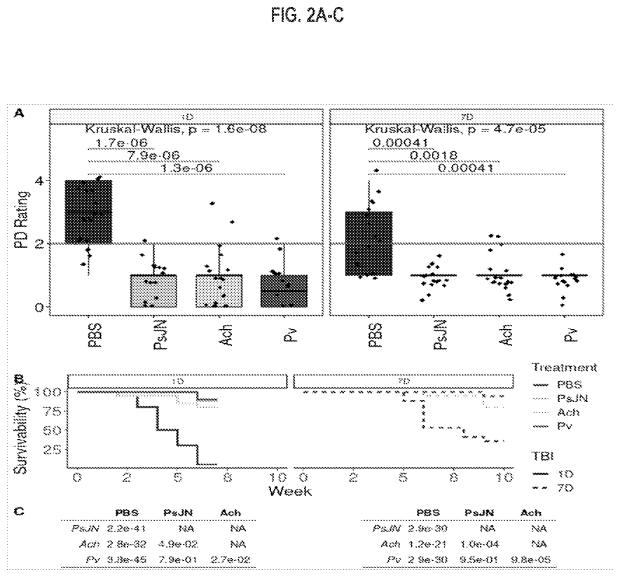

Pre-treatment with biocontrol agents reduces PD symptoms. PD symptom development, including scorched leaves, began 10 weeks after the plants were inoculated with the pathogen. Disease symptom severity was significantly reduced for all vines inoculated with each biocontrol agent compared to buffer control (FIG. 2A; pairwise Wilcoxon: P<0.05). By the end of the disease bioassay, the vines pre-treated with 1×PBS and challenged with X. fastidiosa 1 or 7 days later exhibited average disease ratings of 2.9 and 2.12, respectively based on the 0 to 5 disease progression scale mentioned above. Vines pre-treated with Pa. phytofirmans and challenged with X. fastidiosa 1 or 7 days later exhibited average disease ratings of 0.67 and 0.26, respectively. Vines pre-treated with the Achromobacter vitis and challenged with X. fastidiosa 1 or 7 days later exhibited average disease ratings of 0.85 and 0.32, respectively. Finally, vines pre-treated with the Pseudomonas viridiflava and challenged with X. fastidiosa 1 or 7 days later exhibited average disease ratings of 0.6 and 0.23, respectively.

In addition, the inventors performed a vine survival assay. A visual rating of 2 was considered a critical point in terms of disease symptom development, because once this visual rating was achieved, the disease progression was considered fatal. By the end of the disease bioassay, 44 plants were given a fatal rating of 2 or greater across both time points of X. fastidiosa challenge. Thirty of these vines occurred in control vines pre-treated with 1×PBS buffer, 8 in the Achromobacter vitis pre-treated vines, 3 in Pa. phytofirmans pre-treated vines and 3 in the Pseudomonas viridiflava pre-treated vines. Therefore, vines inoculated with any of the biocontrol agents had greater than 75% survival probability at the end of the bioassay compared to control vines which had less than a 10% survival at the end of the bioassay (FIG. 2B; pairwise Wilcoxon: P<0.05). Pairwise log-rank tests confirmed that the Pa. phytofirmans, Pseudomonas viridiflava, and Achromobacter vitis all had significantly higher survival rates compared to the 1×PBS negative buffer control, regardless of the timing of X. fastidiosa challenge (FIG. 2B; pairwise Log-Rank test: p<0.0001). Between biocontrol agents, Achromobacter vitis had a decreased survival rate compared to the other two biocontrols regardless of the timing of a X. fastidiosa challenge (FIG. 2B; pairwise Log-Rank test: (p<0.05). Cox proportional hazard analysis, a survival analysis regression model, showed the Pa. phytofirmans, Pseudomonas viridiflava, and Achromobacter vitis each confer a significant reduction in risk of death when inoculated with X. fastidiosa regardless of the timing X. fastidiosa challenge (FIG. 2C; Cox Proportional hazards; p<0.001).

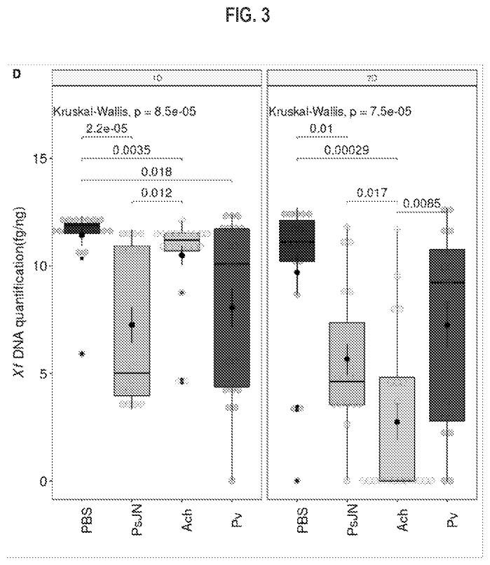

Pre-treatment with biocontrol agents reduced X. fastidiosa titer. For vines challenged with X. fastidiosa 1 day after pre-treatment, all vines inoculated with the Pa. phytofirmans, Achromobacter vitis, and Pseudomonas viridiflava showed a significant reduction in average pathogen titer (7.25, 10.5, and 8.06 log X. fastidiosa DNA quantification per total DNA extracted, respectively) compared to the average pathogen titer found in the 1×PBS buffer pre-treated vines (11.44 log X. fastidiosa DNA quantification) (FIG. 3; pairwise-Wilcoxon: p<0.05). In addition, vines pre-treated with Pa. phytofirmans exhibited significantly lower pathogen titer compared to vines pre-treated with the Achromobacter vitis, which had the highest average pathogen titer (FIG. 3; pairwise-Wilcoxon: p<0.05). For vines challenged with X. fastidiosa 7 days after pre-treatment, only vines pre-treated with Pa. phytofirmans and Achromobacter vitis showed a significant reduction in average pathogen titer (5.66 and 2.74 log X. fastidiosa DNA quantification, respectively) compared to the average pathogen titer found in the sterile buffer pretreated vines (9.70 log X. fastidiosa DNA quantification) (FIG. 3; pairwise-Wilcoxon: p<0.01). At the 7 day X. fastidiosa challenge, Pseudomonas viridiflava inoculated vines did not exhibit less pathogen titer (7.24 log X. fastidiosa DNA quantification) and showed the largest variation in X. fastidiosa titer, with some titers exceeding the largest titers associated with 1×PBS buffer pre-treated vines (FIG. 3; pairwise-Wilcoxon). In contrast to the 1-day X. fastidiosa challenged vines, Achromobacter vitis pre-treated vines exhibited significantly less average pathogen titer compared to both Pa. phytofirmans and Pseudomonas viridiflava pre-treated vines (FIG. 3; pairwise-Wilcoxon: p<0.05).

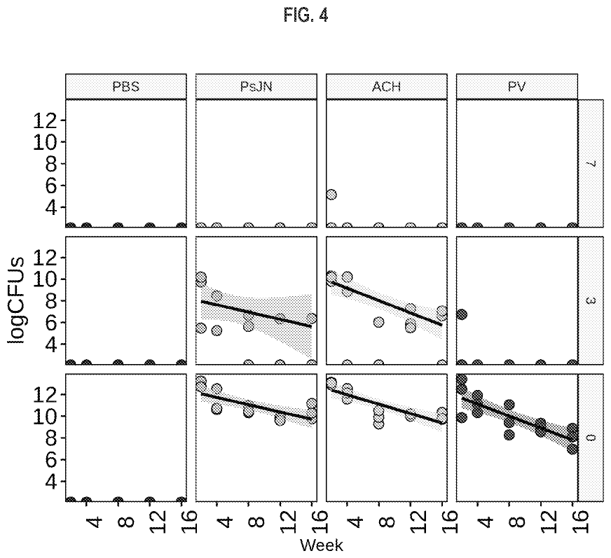

Biocontrol agents persist in the vines over 16 weeks and remain largely localized to the point of inoculation. Each biocontrol agent persisted in the vines throughout the 16-week experiment and the vines showed no decline in health indicating the biocontrol agents do not have a detrimental effect on the plant. Population sizes of the Pa. phytofirmans, Pseudomonas viridiflava, and Achromobacter vitis were quantified at the point of inoculation, three internodes above the point of inoculation, and seven internodes above the point of inoculation on mature grape stems (FIG. 4). Endophytes were not detected seven internodes above the point of inoculation throughout the 16-week growth period, with the exception of one sample at the two-week time point inoculated with the Achromobacter vitis and Pa. phytofirmans was recovered from the point of inoculation at the point of inoculation (10⁶ CFU/g stem tissue) up until four weeks post inoculation. However, Pa. phytofirmans was only recovered at 3 internodes above the point of inoculation (10⁵ CFU/g stem tissue) at two weeks post inoculation. Following this, it declined in titer to ~200 CFU/g stem tissue for all subsequent time points. Achromobacter vitis was recovered from the point of inoculation at an average titer of 10⁶ CFU/g stem tissue at the point of inoculation and 3 internodes above the point of inoculation at 10⁵ CFU/g stem tissue up until four weeks post inoculation. Subsequent time points indicate that Achromobacter vitis colonized the point of inoculation at 10⁵ CFU/g stem tissue and three nodes above the point of inoculation at ~500 CFU/g stem tissue. Pseudomonas viridiflava declined in titer over the 16-week assay at the point of inoculation and was only found three internodes above the point of inoculation (at an average concentration of <300 CFU/g stem tissue) at the two-week time point post inoculation and not at subsequent time points.

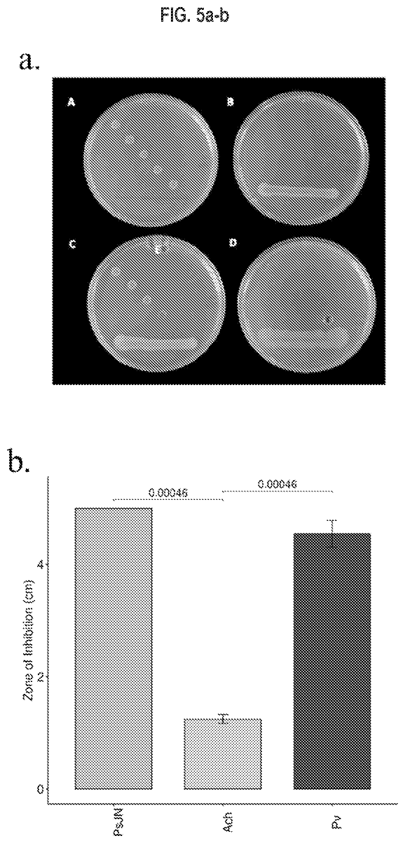

Antibiosis activity of the biocontrol agents. One mechanism found in successful biocontrol agents is the production of antibiotics that inhibit or kill target pathogens. The inventors assessed these novel biocontrol agents for their ability to inhibit the growth of X. fastidiosa on solid PD3 medium (FIG. 5). Inhibition was assessed by calculating the percentage of growth of X. fastidiosa along a five cm distance. Achromobacter vitis exhibited an inhibitory effect on the growth of X. fastidiosa in comparison to the negative control, which was observed on average after 1.24 cm±0.24 from the Achromobacter vitis per replicate. Pa. phytofirmans, the positive control, and Pseudomonas viridiflava exhibited more pronounced inhibition of X. fastidiosa growth. No X. fastidiosa growth was observed within five cm of Pa. phytofirmans in every replicate and X. fastidiosa growth was only observed on average after 4.54 cm±0.72 from Pseudomonas viridiflava per replicate.

### Primer Sequences:

fastidiosa ITS primers: XfITSF6
SEQ ID NO: 1X
GAGTATGGTGAATATAATTGTC
fastidiosa ITS primers: XfITSR6
SEQ ID NO: 2X
CAACATAAACCCAAACCTAT
16S universal primers: 16S U1
SEQ ID NO: 3
CCAGCAGCCGCGGTAATACG
16S universal primers: 16S U2
SEQ ID NO: 4
ATCGGCTACCTTGTTACGACTTC

### V. Complete Citation of References Cited Above by Author and Year

1. Ali M. A., Lou Y., Hafeez R., Li X., Hossain A., Xie T., et al. 2021. Functional analysis and genome mining reveal high potential of biocontrol and plant growth promotion in nodule-inhabiting bacteria within Paenibacillus polymyxa complex. Front Microbiol. 11 doi:10.3389/fmicb.2020.618601
2. Arkin A. P., Cottingham R. W., Henry C. S., Harris N. L., Stevens R. L., Maslov S., et al. 2018. KBase: the United States Department of Energy systems biology knowledgebase. Nat. Biotechnol. 36:566-569.
3. Blin K., Shaw S., Kloosterman A. M., Charlop-Powers Z., van Wezel G. P., Medema M. H., et al. 2021. antiSMASH 6.0: improving cluster detection and comparison capabilities. Nucleic Acids Res. 49:W29-W35.
4. Bolger A. M., Lohse M., and Usadel B. 2014. Trimmomatic: a flexible trimmer for Illumina sequence data. Bioinformatics 30:2114-2120.
5. Chaumeil P-A., Mussig A. J., Hugenholtz P., and Parks D. H. 2019. GTDB-Tk: a toolkit to classify genomes with the genome taxonomy database. Bioinformatics 36:1925-1927.
6. Daugherty M. P., O'Neill S., Byrne F., and Zeilinger A. 2015. Is vector control sufficient to limit pathogen spread in vineyards? Environ Entomol. 44:789-797.
7. Deyett E., Roper M. C., Ruegger P., Yang J-I., Borneman J., and Rolshausen P. E. 2017. Microbial landscape of the grapevine endosphere in the context of Pierce's disease. Phytobiomes Journal 1:138-149.
8. Gurevich A., Saveliev V., Vyahhi N., Tesler G. 2013. QUAST: quality assessment tool for genome assemblies. Bioinformatics 29:1072-1075.
9. Krivanek A. F., Riaz S., and Walker M. A. 2006. Identification and molecular mapping of PdR1, a primary resistance gene to Pierce's disease in Vitis. Theor Appl Genet. 112:1125-1131.
10. Letunic I., and Bork P. 2019. Interactive Tree Of Life (iTOL) v4: recent updates and new developments. Nucleic Acids Res. 47:W256-W259.
11. Moretti M., Gilardi G., Gullino M. L. and Garibaldi A. 2008. Biological control potential of Achromobacter xylosoxydans for suppressing Fusarium wilt of tomato. Int. J. Bot. 4:369-375.
12. Niem J. M., Billones-Baaijens R., Stodart B., and Savocchia S. 2020. Diversity profiling of grapevine microbial endosphere and antagonistic potential of endophytic Pseudomonas against grapevine trunk diseases. Front. Microbiol. 11: doi: 10.3389/fmicb.2020.00477
13. Seemann T. 2014. Prokka: rapid prokaryotic genome annotation. Bioinformatics 30:2068-2069.
14. Shaffer M., Borton M. A., McGivern B. B., Zayed A. A., La Rosa S. L., Solden L. M., et al. 2020. DRAM for Distilling Microbial Metabolism to automate the curation of microbiome function. Nucleic Acids Res. 48:8883-8900.
15. Wick R. R., Judd L. M., Gorrie C. L., and Holt K. E. 2017. Completing bacterial genome assemblies with multiplex MinION sequencing. Microb. Genom. 3(10): e000132
16. Wick R. R., Judd L. M., Gorrie C. L., and Holt K. E. 2017. Unicycler: Resolving bacterial genome assemblies from short and long sequencing reads. PLoS Comput. Biol. 13(6): e1005595.

### VI. Exemplary Embodiments

Exemplary embodiments provided in accordance with the presently disclosed subject matter include, but are not limited to, the following embodiments:

Embodiment 1. A method for preventing or treating a Xylella sp. infection in a plant comprising inoculating the plant with Achromobacter vitis, deposited under American Type Culture Collection (ATCC) Patent Deposit No. PTA-127561.

Embodiment 2. The method of embodiment 1, wherein the Xylella sp. is Xylella fastidiosa.

Embodiment 3. The method of embodiment 1 or 2, wherein the plant is a monocotyledonous plant.

Embodiment 4. The method of embodiment 1 or 2, wherein the plant is a dicotyledonous plant.

Embodiment 5. The method of embodiment 1 or 2, wherein the plant is selected from the group consisting of grape, oleander, oak, almond, peach, pear, citrus, coffee, maple, apricot, plum, blackberry, mulberry, olive, elm, sycamore, alfalfa, sweetgum, redbud, hibiscus, catharantus, jacaranda, magnolia, daylily, crepe myrtle, dogwood, maidenhair, and Chitalpa tashkentensis plant.

Embodiment 6. The method of embodiment 5, wherein the plant is a grape plant, an oleander plant, a coffee plant, an alfalfa plant, a peach plant, an olive plant, or a citrus plant.

Embodiment 7. The method of embodiment 6, wherein the plant is a grape plant.

Embodiment 8. The method of any one of embodiments 1-7, wherein the Achromobacter vitis is formulated with at least one carrier when inoculating into a plant.

Embodiment 9. The method of embodiment 8, wherein the carrier is a surface-active agent.

Embodiment 10. The method of any one of embodiments 1-9, wherein inoculating comprises introducing the Achromobacter vitis into the plant.

Embodiment 11. The method of embodiment 10, wherein inoculating comprises introducing the Achromobacter vitis into xylem of the plant.

Embodiment 12. The method of embodiment 10, wherein inoculating comprises introducing the Achromobacter vitis into the plant by injection, spraying, misting, dusting, or topical administration to the plant.

Embodiment 13. The method of embodiment 12, wherein the Achromobacter vitis is injected into the plant.

Embodiment 14. The method of embodiment 12, wherein the Achromobacter vitis is sprayed into the plant.

Embodiment 15. The method of any one of embodiments 1-14, wherein the Achromobacter vitis is inoculated into the plant before a symptom of Xylella sp infection appears in the plant.

Embodiment 16. The method of any one of embodiments 1-14, wherein the Achromobacter vitis is inoculated into the plant after a symptom of Xylella sp infection appears in the plant.

Embodiment 17. The method of embodiment 15 or 16, wherein the symptom of Xylella sp infection is selected from the group consisting of leaf scorch, leaf chlorosis, gum-like substance on leave, withering and desiccation of branches, dwarfing or lack of growth of the plant, drooping appearance and shorter internodes, shriveled fruits on infected plants, premature fruit abscission, hardening and size reduction of fruits, wilting of foliage, and browning to dieback.

Embodiment 18. The method of embodiment 15 or 16, wherein the plant is a grape plant and the symptom of Xylella sp infection is selected from the group consisting of leaf scorch, yellowing around the leaf margins or between the veins, less vigorous and smaller leaf, and patches of brown and green tissue (green islands) on stems.

Embodiment 19. The method of any one of embodiments 1-18, wherein the method further comprises inoculating the plant with a biological or chemical agent that inhibits Xylella fastidiosa infection.

Embodiment 20. The method of embodiment 19, wherein the agent comprises a microorganism, an extract prepared from a microorganism, a pesticide, a bacteriophage, an antibiotic drug, zinc, iron, copper, molybdenum, a derivative thereof, or a combination thereof.

Embodiment 21. A method of preparing an environment for growth of a plant susceptible to Xylella sp. infection comprising inoculating the environment in which the plant grows with Achromobacter vitis.

Embodiment 22. The method of embodiment 21, wherein inoculating comprises spraying, misting, or dusting the Achromobacter vitis into or on soil in which the plant grows, or introducing the Achromobacter vitis into irrigation water that is applied to the plant.

Embodiment 23. The method of embodiment 21 or 22, wherein the Achromobacter vitis Achromobacter vitis is deposited under ATCC Patent Deposit No. PTA-127561.

Embodiment 24. The method of any one of embodiments 21-23, wherein the Xylella sp. is Xylella fastidiosa.

Embodiment 25. The method of any one of embodiments 21-24, wherein the plant is selected from the group consisting of grape, oleander, oak, almond, peach, pear, citrus, coffee, maple, apricot, plum, blackberry, mulberry, olive, elm, sycamore, alfalfa, sweetgum, redbud, hibiscus, catharantus, jacaranda, magnolia, daylily, crepe myrtle, dogwood, maidenhair, and Chitalpa tashkentensis plant.

Embodiment 26. The method of embodiment 25, wherein the plant is a grape plant, an oleander plant, a coffee plant, an alfalfa plant, a peach plant, an olive plant, or a citrus plant.

Embodiment 27. The method of embodiment 26, wherein the plant is a grape plant.

Embodiment 28. A composition for treating or preventing a Xylella sp. infection in a plant comprising Achromobacter vitis, deposited under ATCC Patent Deposit No. PTA-127561, and at least one carrier formulated for delivery to the plant at a concentration ranging from about 104 colony forming units/ml (CFU/ml) to about 108 CFU/ml.

Embodiment 29. The composition of embodiment 28, wherein the carrier is selected from the group consisting of a surface-active agent, an inert material, a preservative, a humectant, a feeding stimulant, an attractant, an encapsulating agent, a binder, an emulsifier, a dye, a UV protective, a buffer, a flow agent, a liposome, a dispersion medium, a micronutrient donor, and the like.

Embodiment 30. The composition of embodiment 29, wherein the surface-active agent is an anionic agent, a cationic agent, or a non-ionic agent.

Embodiment 31. The composition of embodiment 30, wherein the anionic surface-active agent is selected from the group consisting of a carboxylate of a metal; carboxylate of a long chain fatty acid; an N-acylsarcosinate; mono or di-esters of phosphoric acid with fatty alcohol ethoxylates or salts of such esters; sodium dodecyl sulfate; sodium octadecyl sulfate; sodium cetyl sulfate; ethoxylated fatty alcohol sulfates; ethoxylated alkylphenol sulfates; lignin sulfonates; petroleum sulfonates; alkyl-benzene sulfonates; butyl-naphthalene sulfonate; salts of sulfonated naphthalene-formaldehyde condensates; salts of sulfonated phenol-formaldehyde condensates; the amide sulfonates; and the dialkyl sulfosuccinates.

Embodiment 32. The composition of embodiment 30, wherein the cationic surface-active agent is selected from the group consisting of an aliphatic monoamine, an aliphatic diamine, an aliphatic polyamine; oxygen-containing amine; and an amide-linked amine prepared by the condensation of a carboxylic acid with a di- or polyamine; or a quaternary ammonium salt.

Embodiment 33. The composition of embodiment 30, wherein the non-ionic surface-active agent is selected from the group consisting of condensation products of fatty acid esters, fatty alcohols, fatty acid amides, fatty-alkyl- or alkenyl-substituted phenols with ethylene oxide, fatty esters of polyhydric alcohol ethers, condensation products of such esters with ethylene oxide, block copolymers of ethylene oxide and propylene oxide, acetylenic glycols, and ethoxylated acetylenic glycols.

Embodiment 34. The composition of embodiment 29, wherein the inert material is selected from the group consisting of inorganic minerals, phyllosilicates, carbonates, sulfates, phosphates, botanical materials, peanut hulls, rice hulls, and walnut shells.

Embodiment 35. The composition of any one of embodiments 28-34, wherein the composition further comprises a biological or chemical agent which inhibits Xylella fastidiosa infection.

Embodiment 36. The composition of embodiment 35, wherein the agent comprises a microorganism, an extract of a microorganism, a pesticide, a bacteriophage, an antibiotic drug, zinc, iron, copper, molybdenum, a derivative thereof, or a combination thereof.

Embodiment 37. An in vitro culture comprising isolated Achromobacter vitis, deposited under ATCC Patent Deposit No. PTA-127561 and synthetic growth media.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for the subject matter for which they are specifically cited.

What is claimed is:

1. A method for preventing or treating a Xylella sp. infection in a plant comprising inoculating the plant with Achromobacter vitis, deposited under American Type Culture Collection (ATCC) Patent Deposit No. PTA-127561.

2. The method of claim 1, wherein the Xylella sp. is Xylella fastidiosa.

3. The method of claim 1 or 2, wherein the plant is a monocotyledonous plant.

4. The method of claim 1 or 2, wherein the plant is a dicotyledonous plant.

5. The method of claim 1 or 2, wherein the plant is selected from the group consisting of grape, oleander, oak, almond, peach, pear, citrus, coffee, maple, apricot, plum, blackberry, mulberry, olive, elm, sycamore, alfalfa, sweetgum, redbud, hibiscus, catharantus, jacaranda, magnolia, daylily, crepe myrtle, dogwood, maidenhair, and Chitalpa tashkentensis plant.

6. The method of claim 5, wherein the plant is a grape plant, an oleander plant, a coffee plant, an alfalfa plant, a peach plant, an olive plant, or a citrus plant.

7. The method of claim 6, wherein the plant is a grape plant.

8. The method of any one of claims 1-7, wherein the Achromobacter vitis is formulated with at least one carrier when inoculating into a plant.

9. The method of claim 8, wherein the carrier is a surface-active agent.

10. The method of any one of claims 1-9, wherein inoculating comprises introducing the Achromobacter vitis into the plant.

11. The method of claim 10, wherein the inoculating comprises introducing the Achromobacter vitis into xylem of the plant.

12. The method of claim 10, wherein inoculating comprises introducing the Achromobacter vitis into or onto the plant by injection, spraying, misting, dusting, or topical administration to the plant.

13. The method of claim 12, wherein the Achromobacter vitis is injected into the plant.

14. The method of claim 12, wherein the Achromobacter vitis is sprayed onto the plant.

15. The method of any one of claims 1-14, wherein the Achromobacter vitis is inoculated into the plant before at least one Xylella sp infection symptoms appears in the plant.

16. The method of any one of claims 1-14, wherein the Achromobacter vitis is inoculated into the plant after at least one Xylella sp infection symptoms appears in the plant.

17. The method of claim 15 or 16, wherein the at least one Xylella sp infection symptom is selected from the group consisting of leaf scorch, leaf chlorosis, gum-like substance on leave, withering and desiccation of branches, dwarfing or lack of growth of the plant, drooping appearance and shorter internodes, shriveled fruits on infected plants, premature fruit abscission, hardening and size reduction of fruits, wilting of foliage, and browning to dieback.

18. The method of claim 15 or 16, wherein the plant is a grape plant and the at least one Xylella sp infection symptom is selected from the group consisting of leaf scorch, yellowing around the leaf margins or between the veins, less vigorous and smaller leaf, and patches of brown and green tissue (green islands) on stems.

19. The method of any one of claims 1-18, wherein the method further comprising inoculating the plant with a biological or chemical agent that inhibits Xylella fastidiosa infection.

20. The method of claim 19, wherein the agent comprises a microorganism, an extract prepared from a microorganism, a pesticide, a bacteriophage, an antibiotic drug, zinc, iron, copper, molybdenum, a derivative thereof, or a combination thereof.

21. A method of preparing an environment for growth of a plant susceptible to Xylella sp. infection comprising inoculating the environment in which the plant grows with Achromobacter vitis.

22. The method of claim 21, wherein inoculating comprises spraying, misting, or dusting the Achromobacter vitis into or onto soil in which the plant grows, or introducing the Achromobacter vitis into irrigation water that is applied to the plant.

23. The method of claim 21 or 22, wherein the Achromobacter vitis is deposited under ATCC Patent Deposit No. PTA-127561.

24. The method of any one of claims 21-23, wherein the Xylella sp. is Xylella fastidiosa.

25. The method of any one of claims 21-24, wherein the plant is selected from the group consisting of grape, oleander, oak, almond, peach, pear, citrus, coffee, maple, apricot, plum, blackberry, mulberry, olive, elm, sycamore, alfalfa, sweetgum, redbud, hibiscus, catbarantus, jacaranda, magnolia, daylily, crepe myrtle, dogwood, maidenbair, and Chitalpa tashkentensis plant.

26. The method of claim 25, wherein the plant is a grape plant, an oleander plant, a coffee plant, an alfalfa plant, a peach plant, an olive plant, or a citrus plant.

27. The method of claim 26, wherein the plant is a grape plant.

28. A composition for treating or preventing a Xylella sp. infection in a plant comprising Achromobacter vitis, deposited under ATCC Patent Deposit No. PTA-127561, and at least one carrier formulated for delivery to the plant at a concentration ranging from about 10⁴ colony forming units/ml (CFU/ml) to about 10⁸ CFU/ml.

29. The composition of claim 28, wherein the carrier is selected from the group consisting of a surface-active agent, an inert material, a preservative, a humectant, a feeding stimulant, an attractant, an encapsulating agent, a binder, an emulsifier, a dye, a UV protective, a buffer, a flow agent, a liposome, a dispersion medium, and a micronutrient donor.

30. The composition of claim 29, wherein the surface-active agent is an anionic agent, a cationic agent, or a non-ionic agent.

31. The composition of claim 30, wherein the anionic surface-active agent is selected from the group consisting of a carboxylate of a metal; carboxylate of a long chain fatty acid; an N-acylsarcosinate; mono or di-esters of phosphoric acid with fatty alcohol ethoxylates or salts of such esters; sodium dodecyl sulfate; sodium octadecyl sulfate; sodium cetyl sulfate; ethoxylated fatty alcohol sulfates; ethoxylated alkylphenol sulfates; lignin sulfonates; petroleum sulfonates; alkyl-benzene sulfonates; butyl-naphthalene sulfonate; salts of sulfonated naphthalene-formaldehyde condensates; salts of sulfonated phenol-formaldehyde condensates; amide sulfonates; and dialkyl sulfosuccinates.

32. The composition of claim 30, wherein the cationic surface-active agent is selected from the group consisting of an aliphatic monoamine, an aliphatic diamine, an aliphatic polyamine; oxygen-containing amine; and an amide-linked amine prepared by the condensation of a carboxylic acid with a di- or polyamine or a quaternary ammonium salt.

33. The composition of claim 30, wherein the non-ionic surface-active agent is selected from the group consisting of condensation products of fatty acid esters, fatty alcohols, fatty acid amides, fatty-alkyl- or alkenyl-substituted phenols with ethylene oxide, fatty esters of polyhydric alcohol ethers, condensation products of such esters with ethylene oxide, block copolymers of ethylene oxide and propylene oxide, acetylenic glycols, and ethoxylated acetylenic glycols.

34. The composition of claim 29, wherein the inert material is selected from the group consisting of inorganic minerals, phyllosilicates, carbonates, sulfates, phosphates, botanical materials, peanut hulls, rice hulls, and walnut shells.

35. The composition of any one of claims 28-34, wherein the composition further comprises a biological or chemical agent which inhibits Xylella fastidiosa infection.

36. The composition of claim 35, wherein the agent comprises a microorganism, an extract of a microorganism, a pesticide, a bacteriophage, an antibiotic drug, zinc, iron, copper, molybdenum, a derivative thereof, or a combination thereof.

37. An in vitro culture comprising isolated Achromobacter vitis, deposited under ATCC Patent Deposit No. PTA-127561, and synthetic growth media.


SEQUENCE LISTING (4 sequences)

SEQ ID NO: 1 - DNA, 22 bp, synthetic construct
  source 1..22: mol_type = other DNA
gagtatggtgaatataattgtc

SEQ ID NO: 2 - DNA, 20 bp, synthetic construct
  source 1..20: mol_type = other DNA
caacataaacccaaacctat

SEQ ID NO: 3 - DNA, 20 bp, synthetic construct
  source 1..20: mol_type = other DNA
ccagcagccgcggtaatacg

SEQ ID NO: 4 - DNA, 23 bp, synthetic construct
  source 1..23: mol_type = other DNA
atcggctaccttgttacgacttc